(19) United States
(12) Patent Application Publication
(10) Pub. No.: US 2008/0020067 A1
(43) Pub. Date: Jan. 24, 2008

(54) Skin Moisturizer

(76) Inventors: Hirofumi Koda, Osaka (JP); Yoshinobu Kiso, Osaka (JP); Takehiro Matsumoto, Osaka (JP)

Correspondence Address: DRINKER BIDDLE & REATH (DC), 1500 K STREET, N.W., SUITE 1100, WASHINGTON, DC, 20005-1209, US

(21) Appl. No.: 10/594,900

(22) PCT Filed: Mar. 31, 2005
(86) PCT No.: PCT/JP05/06324

§ 371 Date: Aug. 1, 2007

(30) Foreign Application Priority Data
Mar. 31, 2004 (JP) 2004-101734

(51) Int. Cl. A61K 36/31; A61Q 19/00

(52) U.S. Cl. 424/725

(57) ABSTRACT

The present invention provides an oral skin moisturizer that is readily available to those who are suffering from dry skin, has no side effects, and when orally ingested, enhances the moisture-retaining ability of the skin. The invention also provides food products or the like that contain the oral skin moisturizer. Specifically, the oral skin moisturizer contains, as an active ingredient, a extract of a plant of the genus Lepidium of the family Cruciferae. More specifically, the oral skin moisturizer contains as an active ingredient a extract of maca (Lepidium meyenii Walp). Food products, liquors, functional food products, and pharmaceutical products containing the oral skin moisturizer are also provided.

### TECHNICAL FIELD

The present invention relates to skin moisturizers, and in particular, to a skin moisturizer that contains extracts of plants of the genus Cruciferae, in particular, maca plant. The present invention also relates to an aesthetic food composition for improving the ability of the skin to retain moisture.

### BACKGROUND ART

Japanese spend more and more time in low humidity environments, mainly due to the widespread use of air conditioners and tendency to build tightly sealed houses. As a result, an increasing number of people are suffering from dry skin that results in the loss of the important skin barrier function that protects against surrounding environment. As a result, various skin diseases may arise, including atopic dermatitis. Dry skin has therefore become an important health issue.

Two common approaches to the problem of dry skin are the use of external cosmetics and ointments that contain moisturizers and the use of bath agents that provide the skin with necessary moisture and oil. These approaches, however, involve direct exposure of the skin to the skin care products, so that the components other than the active ingredients used in the products, such as cosmetic components, ointment bases and bath agents, inevitably cause skin irritation and inflammation. It has also been found that the formation of the artificial barrier over the skin surface to avoid water loss can inhibit the lipid secretion into intercellular spaces and the activation of lipid synthesis (Non-Patent Document 1).

Certain functional food products have been developed that contain ceramides, aesthetically effective compounds that help to keep the skin moist, provide skin protection, and prevent rough dry skin and wrinkles when orally ingested (Patent Document 1). Since the food products are intended for oral ingestion, they are expected to circumvent the above-described problems. Nevertheless, materials that can be used to make ceramides and are suitable for use in food products are limited. In addition, these materials require special techniques for extraction. It is thus considered difficult to develop ceramide-containing food products that allow people with dry skin to safely ingest ceramides in amounts effective in keeping the skin moist and providing protection for skin. Moreover, these orally ingested ceramide-containing food products cannot achieve sufficient improvement in the moisture-retaining ability of the skin to compensate for the associated cost.

Thus, despite the increasing number of people who are suffering from dry skin because of the even drier environment that they live in, none of the currently available solutions, including external cosmetics, ointments and bath agents, are decisive.

Maca (Lepidium meyenii Walp) is a plant of the genus cruciferae (brassicaceae) that is originally grown in highlands of Peruvian Andes. The plant grows as it crawls on the ground surface and has roots that resemble a radish. Historically, maca has been cultivated in the Andes region for about two thousand years and has been eaten for the purpose of promoting health. It primarily contains polysaccharides and proteins and is also a rich source of various amino acids, including essential amino acids, which cannot be synthesized in our body and must therefore be taken from food. Vitamins (e.g., vitamin B, C and E) and minerals (e.g., calcium, iron and zinc) are also abundant in maca plants. In Peru, maca is much appreciated health-promoting diet and is used in a wide variety of food products, ranging from cookies and a beverage known as “CHICHA DE MACA” to maca liquors and powdery sprinkles on yogurt.

It has been long known that maca brings about revitalizing and energizing effects. A composition that contains maca to provide anticancer effects and promote sexual function has been described (Patent Document 2). Another composition contains maca in combination with deer antler in the hope of increasing steroid level in humans (Patent Document 3). It has also been suggested that maca has a potential use as anti-inflammatory or anti-allergy agents (Non-Patent Document 2). Furthermore, an external medication has been proposed that uses maca extracts to enhance skin whitening and keep the skin moist (Patent Document 4).

Patent Document 1: Japanese Patent Laid-Open Publication No. Hei 11-113530

Patent Document 2: U.S. Pat. No. 6,257,995 B1 Publication

Patent Document 3: Published Japanese Translation of PCT application No. 2003-523945

Patent Document 4: Japanese Patent Laid-Open Publication No. 2001-39854

Non-Patent Document 1: Pharmacia, Vol. 34, No. 8 (1998)

Non-Patent Document 2: IGAKU TO SEIBUTSUGAKU (Medicine and Biology), vol. 145, No. 6, p.81-86 (2002)

As described, the various advantageous effects of maca have drawn much attention and new food and beverage products have come out that use maca in the form of dried and crushed products or extracts. Nevertheless, none of these products have been developed, by taking into consideration the potential skin-improving effect of maca that is brought about, by orally ingesting the plant. A cosmetic product intended for external application, the maca extract-containing skin cream proposed in Patent Document 4 inevitably contains certain ingredients to make the cream applicable to the skin, and as a result, it is hard to say that is a fundamental solution. These ingredients can cause skin problems and make the skin cream less attractive as a solution to dry skin.

The fundamental solution to dry skin seems to be achieved by orally ingesting maca products to improve the metabolism of the body and, ultimately, the moisture-retaining ability of the skin. From this point of view, the present inventors have examined the potential skin-improving effect of maca extracts that is brought about by orally ingesting the extracts and found that oral ingestion of maca extracts significantly improves the moisture-retaining ability of the skin. It is this discovery that ultimately led to the present invention.

No previous studies have suggested the possibility of enhancing the moisture-retaining ability of the skin via oral ingestion of maca extracts. In this regard, the present invention is highly unique and unprecedented.

### DISCLOSURE OF THE INVENTION

Problems to be Solved by the Invention

Accordingly, it is an object of the present invention to provide an oral skin moisturizer that is readily available to those who are suffering from dry skin, has no side effects, and when orally ingested, enhances the moisture-retaining ability of the skin. It is another object of the present invention to provide food products or the like that contain the oral skin moisturizer.

Means for Solving the Problems

One essential aspect of the present invention concerns an oral skin moisturizer containing, as an active ingredient, an extract of a plant of the genus Lepidium of the family Cruciferae.

More specifically, the present invention is the oral skin moisturizer, wherein the plant of the genus Lepidium of the family Cruciferae is maca (Lepidiwn meyenii Walp).

Thus, one most essential aspect of the present invention concerns the oral skin moisturizer containing a maca extract as an active ingredient.

More specifically, the present invention is the oral skin moisturizer, wherein the maca extract is obtained by adding an aqueous ethanol solution to a crushed maca product, and by maintaining the mixture at 40 to 85° C. for extraction.

Another aspect of the present invention concerns a food product, liquor, functional food product, and pharmaceutical product that contain the above-described oral skin moisturizer.

Effect of the Invention

According to the present invention, there is provided an oral skin moisturizer that can improve the moisture-retaining ability of the skin when orally ingested. Although several approaches, including external cosmetics and ointments and bath agents, have been proposed to prevent dry skin, which affect an increasing number of people in the modern low humidity environment, none of them have provided the ultimate solution. Intended for oral ingestion, the oral skin moisturizer of the present invention significantly enhances the moisture-retaining ability of the skin, thus offering the decisive approach to dry skin.

In addition, the oral skin moisturizer of the present invention, prepared from natural maca extracts, causes no adverse side effects and is safe for use.

Best Mode for Carrying Out the Invention

As described above, the present invention provides an oral skin moisturizer that contains extracts of plants of the genus Lepidium of the family Cruciferae. A preferred plant of the genus Lepidium of the family Cruciferae is maca (Lepidium meyenii Walp). Maca is a root vegetable cultivated or naturally growing in highlands of Peruvian Andes at altitudes of 4000 to 5000 m. The roots of maca have been eaten since ancient time, by local peoples.

The present invention will now be described with reference to maca (Lepidium meyenii Walp) as a representative plant of the genus Lepidium of the family Cruciferae.

The extracts of plants of the genus Lepidium of the family Cruciferae for use in the present invention can be obtained from any of the plant's parts, including whole maca plants, flowers, fruits, leaves, stems and subterranean stems and bulbs. Of the different plant parts, bulbs are preferably used in the extraction process using an extraction solvent. When desired, the plant materials are dried, crushed, or cut for extraction.

The extraction solvent may be any solvent, including water, organic solvents and mixtures thereof.

Specific examples include lower alcohols, such as methanol, ethanol and butanol; polyols, such as propylene glycol and 1,3-butylene glycol; esters, such as ethyl acetate and amyl acetate; and ketones, such as acetone and methyl ethyl ketone.

Because the extracts are intended for oral ingestion according to the present invention, mixtures of water and ethanol (i.e., aqueous ethanol solutions) are preferred to ensure safety.

The studies conducted by the present inventors have revealed that the amount of ethanol in the aqueous ethanol solution used as the extraction solvent affects the ability of the extracts to enhance the moisture-retaining property of the skin. Thus, it is important to adjust the ratio of ethanol to water in the aqueous ethanol solution to optimize the effect of the extracts. Specifically, the amount of ethanol is from 30% to 100% by volume, and in particular from about 40% to about 99% by volume.

It is also important to adjust the temperature of the extraction solvent during the extraction process to optimize the effect of the extracts. To facilitate the process, the extraction is preferably carried out at lower temperatures than the boiling point of the solvent. Specifically, the temperature of the solvent is maintained at 40 to 100° C., and in particular at about 60 to about 85° C. during the extraction process.

Upon extraction, maca and the solvent may be mixed in any suitable proportions: it is preferred that about 0.3 to about 5,000 parts by weight of the solvent be used for 1 part by weight of maca. In particular, 5 to 100 parts by weight of the solvent is used for 1 part by weight of maca to maximize the efficiency of the extraction process.

While the maca extracts obtained in the form of solution may be directly used as the oral skin moisturizer without further processing, the extracts may be concentrated by evaporating the solvent to make a concentrate, or the extracts may be dried by lyophilization or air-blowing to form a dry powder. The concentrated extracts and dry powders are preferred in terms of stability during storage and portability. The term “maca extracts” as used herein refers to any of these solutions, concentrates and dry powders.

The present invention provides an oral skin moisturizer containing the above-described maca extracts as an active ingredient, as well as food and beverage products, liquors and functional food products containing the oral skin moisturizer. Maca has been eaten in Peru since ancient time and its extracts have been proven to be safe.

The oral skin moisturizer of the present invention may be formulated into various pharmaceutically acceptable oral dosage forms by mixing the maca extracts in an appropriate amount with other optional additives. Examples of such oral dosage forms include tablets, capsules, granules, powders, syrups and extracts.

The oral skin moisturizer according to the present invention may be formulated with various additives and can be prepared by conventional techniques. Any suitable additive according to Japanese Pharmacopoeia may be added, including solid carriers such as starch, lactose, sucrose, mannitol, carboxymethylcellulose, corn starch, and inorganic salts such as magnesium stearate.

Another aspect of the present invention concerns food products, liquors and functional food products that contain the oral skin moisturizer containing the maca extracts as an active ingredient, obtained as described above. The amount of the oral skin moisturizer in the food products, liquors and functional food products may vary depending on the amount of maca extracts used in the oral skin moisturizer. The amount of maca extracts may be properly determined by considering such factors as the desired effect, the flavor and the color of the resulting products. The amount of maca extracts in the oral skin moisturizer (as measured by the dry weight) is typically from 0.01 to 99.9%, preferably from 0.01 to 99.5%.

Examples of the food and beverage products provided by the present invention include candies, troches, chewing gums, yogurt, ice cream, pudding, jelly, sweetened and jellied bean pastes (Mizu Yokan), alcohol beverages, coffee beverages, juice beverages, fruit juice beverages, sodas, soft drinks, milk, whey drinks, lactobacillus drinks and various other food and beverage products.

These food and beverage products can be prepared by conventional techniques and may be formulated with optional additives. Any additive commonly used in food products can be used in the preparation of these food and beverage products. Examples of such additives include glucose, fructose, sucrose, maltose, sorbitol, stevioside, rubsoside, corn syrup, lactose, citric acid, tartaric acid, malic acid, succinic acid, lactic acid, L-ascorbic acid, dl-α-tocopherol, sodium erythorbate, glycerol, propyleneglycol, glycerol fatty acid esters, polyglycerol fatty acid esters, sucrose fatty acid esters, sorbitan fatty acid esters, propylene glycol fatty acid esters, gum arabic, carrageenan, casein, gelatin, pectin, agar, vitamin Bs, nicotinamide, calcium pantothenate, amino acids, calcium salts, pigments, flavors and preservatives.

Examples of liquors, or alcohol beverages, that contain the maca extracts of the present invention include spirits, liqueurs, gin, vodka, tequila, brandy, whiskey, shocyu, wine, and distilled liquors. It has turned out that distilled liquor made by fermenting muscat grape followed by distillation serves as a suitable base alcohol for the maca extracts that can decrease the distinctive smell of maca.

The functional food products may be provided in various forms, such as tablets, capsules, granules and powders.

The maca extracts may be administered at any suitable daily dose. For example, the extracts may be administered at a daily dose of about 0.01 mg to about 10 g as measured by the dry weight. For oral administration, a daily dose of 1 mg to 1,000 mg (as measured by the dry weight) is particularly effective in enhancing the moisture-retaining ability of the skin.

Other health food materials that can enhance the moisture-retaining ability of the skin via oral ingestion, such as collagen and hyaluronic acid, may be used in combination with the maca extracts of the present invention. Flavors, colors, antioxidants and other additives commonly used in food and beverage products may also be added to the maca extracts.

The liquors provided by the present invention may further contain fruit juices, spirits, sugars, herb extracts and other optional ingredients. Examples of fruit juice include juices of strawberry, grapefruit, plum, Kyohou grape, apple, peach, pear, cranberry, melon, lemon and lime. These fruit juices may be used either individually or in combination of two or more.

### EXAMPLES

The present invention will now be described with reference to examples and experiments. These examples, however, are provided by way of example only and are not intended to limit the scope of the invention in any way. Throughout the following examples, “%” means “% by weight” unless otherwise specified.

### Example 1

### Preparation of Maca Extracts

3 kg of a dried and crushed maca product were placed in a stainless steel vessel. To this vessel, 30 L of a 50% (by volume) aqueous ethanol solution were added and the solution was stirred for 3 hours at 60° C. for extraction. The solution was filtered and the solvent was removed from the filtrate to give 150 g of maca extract.

### Example 2

### Preparation of Maca Extracts

3 kg of a dried and crushed maca product were placed in a stainless steel vessel. To this vessel, 30 L of a 99% (by volume) aqueous ethanol solution were added and the solution was stirred for 3 hours at 60° C. The solution was filtered and the solvent was removed from the filtrate to give 180 g of maca extract.

### Example 3

### Oral Skin Moisturizer Containing Maca Extract

(1) Tablets

Using a single stroke tablet machine, 66.7 g of the maca extract obtained in Example 1, 232.0 g of lactose and 0.5 g of magnesium stearate were made into 10 mm tablets each weighing 300 mg.

(2) Powders

0.5 g of magnesium stearate was added to 99.5 g of the maca extracts obtained in Example 1. The mixture was compressed, crushed, sorted and sieved to obtain 20 to 50 mesh granules.

### Example 4

### Food and Beverage Products Containing Maca Extract

Different food and beverage products containing the maca extract obtained in Example 1 and having different compositions were prepared as shown below.

| | |
|---|---|
| (Composition) | (Parts by weight) |

(1) Candy

| | |
|---|---|
| Sorbitol powder | 99.7 |
| Flavor | 0.2 |
| Maca extract | 0.05 |
| Sorbitol seed | 0.05 |
| Total | 100.00 |

(2) Chewing gum

| | |
|---|---|
| Gum base | 20.0 |
| Calcium carbonate | 2.0 |
| Stevioside | 0.1 |
| Maca extract | 0.05 |
| Lactose | 76.85 |
| Flavor | 1.0 |
| Total | 100.00 |

(3) Caramel

| | |
|---|---|
| Granulated sugar | 32.0 |
| Starch syrup | 20.0 |
| Milk powder | 40.0 |
| Hardened oil | 4.0 |
| Table salt | 0.6 |
| Flavor | 0.02 |
| Water | 3.22 |
| Maca extract | 0.16 |
| Total | 100.00 |

(4) Soda

| | |
|---|---|
| Granulated sugar | 8.0 |
| Concentrated lemon juice | 1.0 |
| L-ascorbic acid | 0.10 |
| Citric acid | 0.09 |
| Sodium citrate | 0.05 |
| Coloring agent | 0.05 |
| Carbonated water | 90.70 |
| Maca extract | 0.01 |
| Total | 100.00 |

(5) Juice beverage

| | |
|---|---|
| Frozen concentrated | 5.0 |
| orange juice | |
| High fructose corn syrup | 1.0 |
| Citric acid | 0.10 |
| L-ascorbic acid | 0.09 |
| Maca extract | 0.05 |
| Flavor | 0.20 |
| Coloring agent | 0.10 |
| Water | 93.46 |
| Total | 100.00 |

(6) Lactobacillus beverage

| | |
|---|---|
| Fermented milk | 14.76 |
| (Solid milk component = 21%) | |
| High fructose corn syrup | 13.31 |
| Pectin | 0.50 |
| Citric acid | 0.08 |
| Flavor | 0.15 |
| Water | 71.14 |
| Maca extract | 0.06 |
| Total | 100.00 |

(7) Alcohol beverage

| | |
|---|---|
| 50% ethanol | 32.0 |
| Sugar | 8.2 |
| Fruit juice | 2.4 |
| Maca extract | 0.4 |
| Water | 57.0 |
| Total | 100.0 |

Experiment: Ability of maca extract to enhance moisture-retaining property of skin

Healthy volunteered female subjects were tested for the effect of oral ingestion of maca extract on the moisture-retaining property of the skin.

(1) Subjects

10 females in 30's to 40's

(2) Samples Tested

A “maca-containing liquor” which is maca-containing alcohol beverage (7) prepared in Example 4 and containing the maca extract obtained in Example 1 (50% alcohol extract) was tested. A “maca-free liquor” that has the same composition as the alcohol beverage (7) of Example 4 except that the maca extract was excluded was used as a control.

(3) Method

10 subjects were randomly divided into two groups of five: one group was given the maca-containing liquor while the other group was given the maca-free liquor. Each subject consumed the assigned sample at a dose of 100 mL/day over a two-week period. 400 mg of maca extract was present in 100 mL of maca-containing liquor.

(4) Technique Used to Determine Skin Moisture (Stratum Corneum Moisture) Content

Before and two weeks following the ingestion of the assigned sample, the stratum corneum moisture content of the upper arm skin of each subject was measured using SKICON (IBS Co., Ltd.), an instrument designed to measure stratum corneum moisture content based on the skin conductivity. The principle is that electrolytes in a stratum corneum containing more moisture tend to be a solution state, leading to a higher conductivity. For each subject, five measurements were taken at five different points and average was taken.

The results are shown in FIG. 1. Each data point indicates average of five subjects. For the maca-free liquor group, the stratum corneum moisture content measured after the two-week ingestion period did not differ significantly from the moisture content measured prior to the ingestion period. In contrast, the stratum corneum moisture content for the maca-containing liquor group increased significantly (p<0.05) after the two weeks ingestion period from the moisture content prior to the ingestion period. Thus, it has been demonstrated that the alcohol beverage containing the oral skin moisturizer of the present invention significantly improves the moisture-retaining ability of the skin via oral ingestion.

### INDUSTRIAL APPLICABILITY

As set forth, the oral skin moisturizer containing the maca extract of the present invention as an active ingredient enhances the moisture-retaining ability of the skin when orally ingested, thus serving as an highly effective countermeasure against dry skin.

Although several approaches, including external cosmetics and ointments and bath agents, have been proposed to prevent dry skin, none of them have provided the ultimate solution. Unlike these approaches, the oral skin moisturizer of the present invention is intended for oral ingestion and significantly enhances the moisture-retaining ability of the skin, thus offering the decisive and unprecedented solution to dry skin.

### BRIEF DESCRIPTION OF THE DRAWINGS

FIG. 1 is a diagram showing the results of Test Example of the present invention.

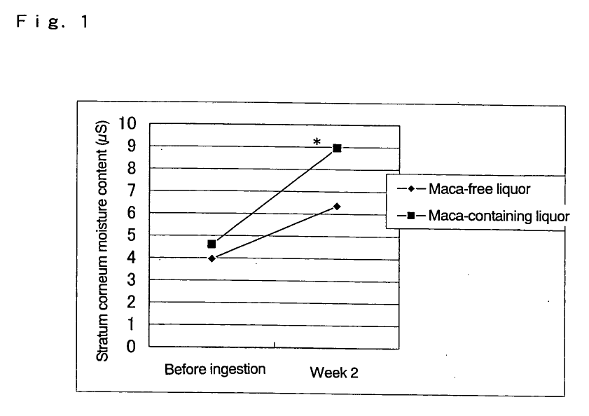

1. An oral skin moisturizer comprising, as an active ingredient, an extract of a plant of the genus Lepidium of the family Cruciferae.

2. The oral skin moisturizer according to claim 1, wherein the plant of the genus Lepidium of the family Cruciferae is maca (Lepidium meyenii Walp).

3. The oral skin moisturizer according to claim 1, containing a maca extract as an active ingredient.

4. The oral skin moisturizer according to claim 3, wherein the maca extract is obtained by adding an aqueous ethanol solution to a crushed maca product, and maintaining the mixture at 40 to 85° C. for extraction.

5. A food product, liquor, functional food product, or pharmaceutical product containing the oral skin moisturizer according to claim 1.
